(19) United States
(12) Patent Application Publication
(10) Pub. No.: US 2014/0087003 A1
(43) Pub. Date: Mar. 27, 2014

(54) METHOD FOR TREATING VENOMOUS BITES AND STINGS

(71) Applicant: Ignacio CISNEROS, Odessa, TX (US)

(72) Inventor: Ignacio CISNEROS, Odessa, TX (US)

(21) Appl. No.: 13/762,118

(22) Filed: Feb. 7, 2013

Related U.S. Application Data
(60) Provisional application No. 61/703,956, filed on Sep. 21, 2012.

(51) Int. Cl. A61K 33/00

(52) U.S. Cl. CPC A61K 33/00
USPC 424/600

(57) ABSTRACT

A method using an alkaline sodium silicate composition to inhibit the toxic effects of venom and treat venomous bites and stings.

### BACKGROUND OF THE INVENTION

1. Field of the Invention

The present invention relates to the treatment of venomous bites and stings, such as from snakes and insects.

2. Description of the Related Art

In the Animal kingdom, a number of venomous animals, such as snakes, scorpions, spiders and jellyfish, produce venom that is harmful to humans, and to their pets and livestock. For humans alone, approximately one million people throughout the world are bitten each year by venomous (poisonous) snakes. It has been estimated that of these some 100,000 die and that another 300,000 will suffer some form of disability for the remainder of their lives. Whereas a toxin, such as produced by bacteria (e.g., botulinum toxin, tetanus toxin, etc.), is a single protein or peptide, venom is a relatively complex mixture of different components, including mixtures of toxins, proteins and peptides. The mechanisms of action of the venoms and the biological reactions of the victim to venoms are quite diverse. Depending on the nature of the venoms, their toxic effects may be harmful to the cardiovascular, hematologic, nervous, and/or respiratory systems.

Notable examples of sources of venom in the Animal kingdom are:

i) Chordata. A number of Chordata classes are sources of venoms (e.g., Amphibians, Fish, Reptiles). Among Reptiles, the most significant order is snakes. Snake venom is a relatively complex mixture of enzymes, non-enzymatic proteins and peptides, and as yet unidentified compounds (Wingert et al., 1975 and Christopher et al., 1986). While there are some chemical similarities, the venom of each species exhibits its own characteristic toxicity (Ellenhorn et al., 1988). Of the over 100 species of snakes in the United States, approximately 10% are poisonous (Parrish, 1966). The majority of these are from the family Crotalidae. The venomous species include the rattlesnakes (Crotalus), cottonmouths and copperheads (Agkistrodon), and pigmy and massassauga rattlesnakes (Sistrurus). There are also poisonous members of the Elapidae family, the coral snakes (Micruroides) (Russell et al., 1975).

ii) Arthropoda. In the Arthropoda phylum, an important class is Arachnida. Among Arachnida, scorpions (Order Scorpiones) produce the most significant venoms. While scorpion venoms are also complex mixtures, there has been some success identifying their active agents. Approximately thirty different protein neurotoxins, each having a molecular weight of about 7000 daltons, have been isolated (Ayeb et al., 1984). Of the approximately 650 scorpion species, the most dangerous belong to the Buthidae family and the genuses Tityus (North and South America), Centruroides (U.S. and Mexico), Centrurus (Mexico), Androctonus (Mediterranean/North Africa), Buthacus (Mediterranean/North Africa), Leiurus (Mediterranean/North Africa), Buthotus (Mediterranean/North Africa), Buthus (Mediterranean/North Africa), and Parabuthus (South Africa) (Hassan, 1984).

iii) Coelenterata. In the Coelenterata phylum, jelly fish are important venomous members; the venom from Chironex fleckeri is among the most potent and medically significant. In the waters off Northern Australia, about one fatality occurs each year (Lumley et al., 1988). Several toxic fractions have been characterized from C. fleckeri venom including two high molecular weight myotoxins (Endean, 1987)) and several low molecular weight toxins having hemolytic or dermonecrotic properties (Olson et al., 1984; Baxter et al., 1969).

iv) Mollusca. In the Mollusca phylum, the most significant venomous members are the coneshells (Conidae) which produce potent myotoxins that can be fatal (Habermehl, 1981). Little is known about the structure of the molluscan myotoxins.

Snake venoms, produced primarily for the procurement of prey or in a defensive role, are complex biological mixtures of upwards of 50 components. Death of prey from a snake bite is due to respiratory or circulatory failure caused by various neurotoxins, cardiotoxins (also called cytotoxins), coagulation factors, and other substances acting alone or synergistically. Snake venoms also contain a number of enzymes which when injected into the prey start tissue digestion. The venoms thus contain substances designed to affect the vital processes such as nerve and muscle function, the action of the heart, circulation of the blood and the permeability of membranes. Most constituents of snake venoms are proteins, but low molecular weight compounds such as peptides, nucleotides and metal ions are also present (Karlsson, 1979).

Poisonous (venomous) snakes may be divided into 4 main families, the Colubridae, the Viperidae, the Hydrophidae and the Elapidae (Tu, 1982). The systematics of these snakes are described in Tables 1 and 2 below. Rattlesnakes which are particular to the American continent are members of a subfamily of venomous snakes from the Viperidae family known as Crotalinae, genera Crotalus or Sistrusus (rattlesnakes), Bothrops, Apkistrodon and Trimerisurus. The two rattlesnake genera may be broken down still further into species and sub species. These snakes are also called the “pit vipers” due to the presence of facial sensory heat pits; however their most prominent feature is the rattle which when present distinguishes them from all other snakes. Each species or subspecies occupies a distinct geographical location in the North or South America. The venom of each species of rattlesnake contains components which may be common to all rattlesnakes, common to only some smaller groups or may be specific to a single species or subspecies (Russell, 1983).

TABLE 1
Classification of venomous snakes

Class: Reptilla (Reptiles)
Order: Squamata (Snakes and Lizards)
Suborder: Serpentes (Snakes)
Infra order: Alethinophidia (Spectacled Snakes)
Superfamily: Colubroidea (Advanced Snakes)

| Family | Subfamily | Tribe |
|---|---|---|
| Colubridae | Nactricinae (Nactricine Water Snakes) | |
| (Colubrid Snakes) | Dispholidinae (African Rear-Fanged Snakes) | |
| | Atrctaspidinae (Burrowing False Vipers) | |
| Elapidae | Bungarinae (Cobras) | Bungarini (Kraits) |
| (Palatine Erectors) | Elapinae | Najini (Cobras) |
| | | Elapini (American Coral) |
| | | Maticorini (Asian Coral) |
| | | Laticaudini (Sea Kraits) |
| Hydrophhiidae | Oxyuraninae (Australasian Venomous Snakes) | Ephalophini |
| (Palatine Draggers) | Hydrophiinae (True Sea Snakes) | Hydrelapini |
| | | Aipysurini |
| | | Hydrophini |
| Viperidae | Viperinae (Pitless Vipers) | Viprini (True Vipers) |
| (Vipers) | | Azemiopini (Fea's Viper) |
| | | Causini (Night Adders) |
| | Crotaline (Pit Vipers) | Lachesini (Bush-masters) |
| | | Crotalini (Viviparous Pit Vipers) |

TABLE 2
Classification and geographical distribution of subfamily Crotalinae.

| Tribe | Genus | Habitat |
|---|---|---|
| Lachesini | Lachesis (Bush-masters) | Central and South America |
| Crotalini | Crotalus (Rattle-snakes) | North, Central and South America |
| | Sistrums (Mass-augas and pigmy rattlesnakes) | North America |
| | Bothrops (New World pit vipers) | Central and North America |
| | Trimeresurus (Asiatic pit vipers) | Asia and North America |
| | Hypnale | Asia |
| | Agkistrodon (Moccasin) | North America, Southeast Europe, and |
| | | Asia |

Venomous bites and stings, particularly venomous snake bites, result in tens of thousands of deaths each year worldwide, primarily in underdeveloped countries. Exposure to most venoms in humans does not result in protective immunity. Furthermore, all attempts to create protective immunity against venoms with vaccines have failed (Russell, 1971). For animal venoms, active immunization has not been feasible. First, many animal venoms are too difficult or too expensive to obtain to immunize a population where a relatively small percentage of that population will be exposed to the animal venom. Second, even if they can be obtained, animal venoms, unless detoxified, may cause more morbidity when administered to a large population than would be caused by the venomous animals themselves. Third, even if the venom is affordable, obtained in sufficient quantity, and detoxified, it is extremely difficult to achieve the titer of circulating antibody necessary to neutralize the infusion of what can be a large amount of venom (up to one gram of animal venom as compared with nanogram or picogram amounts of tetanus toxin). Finally, even with successful immunization, immunological memory is too slow to respond to the immediate crisis of envenomation.

Passive immunization, like active immunization, relies on antibodies binding to antigens. Antivenom refers to antibody raised against whole venom. In the case of passive immunization, the antibody used to bind the venom (antigen) is not made in the animal afflicted with the venom. Generally, an immune response is generated in a first animal. The serum of the first animal is then administered to the afflicted animal to supply a source of specific and reactive antibody. Antivenom is thus the serum or partially purified antibody fraction of serum from animals that have been rendered immune to venom toxicity as a result of a regimen of injections of increasing doses of the venom. The administered antivenom antibody functions to some extent as though it were endogenous antibody, binding the venom toxins and reducing their toxicity.

The first step in treatment by passive immunization involves raising an antibody with reactivity that is specific for the venom. Such an antibody is referred to as an antivenom. As noted above, venoms pose unique problems for immunization. They are often expensive and available in only small amounts. Antivenoms have been raised in a number of mammals (Perez et al., 1984 (mice); Iddon et al., 1988 (mice); Martinez et al., 1989 (mice); Ayeb et al., 1984 (rabbits); Russell et al., 1970 (goats); Curry et al., 1983-1984 (goats); Hassan, 1984 (cows)). Horses, however, are the animal of choice by an overwhelming number of investigators and commercial antivenom producers (World Health Organization Publication No. 58, 1981). Horses are sturdy and tolerant to the antibody-raising process. Most importantly, they yield large volumes of blood (as much as ten liters per bleeding for large animals). However, there are a number of significant disadvantages when using horses for antivenom production. Nevertheless, in spite of these problems, horse antivenom is the primary specific treatment of most venom poisonings. It is considered vital for treating severe cases of snake envenomation (Parrish et al., 1970).

Similarly, horse serum containing antivenoms is considered life-saving in the treatment of scorpion stings (Hassan, 1984). Because the commercial antivenoms presently available can cause their own adverse reactions, the risk of possible death or serious injury from the venom must be weighed against the risk of a hypersensitivity reaction to horse serum. Currently, a large number and diversity of monospecific and polyspecific antivenoms are produced around the world.

U.S. Pat. No. 6,613,326 discloses polyvalent antivenom which was raised against a mixture (“cocktail”) of venoms as immunogen. On the other hand, U.S. Pat. No. 6,833,131 discloses an antivenom that is a mixture of monospecific antisera, each of which were raised separately against a specific venom. These polyvalent antivenoms can be designed according to the potential for envenomation by venomous animals of any particular geographical area.

In the United States, venomous snakes bite approximately 8,000 people each year resulting in significant morbidity and several deaths (8-12 deaths) (Dart and McNally, 2001; Gold et al., 2002; 2004). Envenomation may cause serious problems that can result in severe local tissue damage, functional disability, and loss of extremities. Ninety nine percent of snakebites in the United States are caused by the subfamily Crotalinae, also known as the pit vipers, and which includes rattlesnakes (genera Crotalus and Sistrurus); copperheads (Agkistrodon contortrix), and cottonmouths or water moccasins (Agkistrodon piscivorous) (Smith and Figge, 1991). Envenomation by crotalid snakes generally induces systemic disturbances in hemostasis including spontaneous bleeding and blood incoagulability and strong local effects characterized by edema, ecchymoses, blisters, extensive hemorrhage, and necrosis involving massive degradation of the extracellular matrix at the snakebite area (Warrell, 1996).

In developed countries where access to medical facilities and treatment with antivenom is readily available, death resulting from snake envenomation is rare. Antivenom however frequently causes early anaphylactoid and serum sickness adverse reactions, which could be severe and life threatening (Theakston and Reid, 1983). In addition, the efficacy of antivenom administration against local symptoms has been reported to be poor due to rapid development of the damage at the bitten area (Russell et al., 1973; Ownby et al., 1986; Evans and Ownby, 1999; Rucavado et al., 2000). Although antivenom therapy is largely successful in reducing the mortality associated with venomous snake bites, it is less effective in reducing local hemorrhage and tissue necrosis, prominent symptoms of envenomation. Frequently the local tissue necrosis subsequent to a snake bite may be so severe as to result in permanent disfigurement, impairment or, in extreme cases, loss of an affected extremity.

The venom of poisonous snakes and other venomous animals is a complex mixture containing proteins with many enzymatic activities. Most of local and systemic complications following Crotalinae envenomation are because of the actions of proteases in the venom. Hemorrhagic metalloproteinases play a crucial role in the development of local tissue damage causing hemorrhage, edema, and myonecrosis (Bjarnason and Fox, 1994; Gutierrez and Rucavado, 2000). These hemorrhagic metalloproteinases also play a key role in spreading venom components into the circulatory system (Anai et al., 2002). In addition, serine proteases are also assumed to be responsible for the development of local and systemic complications after envenomation (Braud et al., 2000). It is assumed that the inhibition of venom proteases in an early phase of envenomation may be a promising alternative therapy (Perez and Sánchez, 1999; Rucavado et al., 2000). The hemorrhagic factors of snake venom, which are those enzymes responsible for hemorrhagic activity, have been found to be metal dependent. Elimination of Ca (II), Mg (II) and Zn (II) from these hemorrhagic factors by various metal chelators in vitro eliminates the hemorrhagic activity (Tu, 1977). See also Bjarnason, 1978, Ownby et al., 1975, and Friederick et al., 1971.

A mixture of diethylenetriaminepentaacetic acid (DTPA), a metal chelator, and procaine when injected within 15 minutes of envenomation or ethylenediaminetetraacetic acid (EDTA) when injected within 30 minutes of envenomation by snake in the vicinity of the bite is known to reduce local hemorrhage; however, these agents are without effect in reducing tissue necrosis or lethality (Ownby et al., 1972). Moreover, EDTA is contraindicated in the presence of a disturbed electrolyte balance, a condition not uncommon with many snake bite victims. U.S. Pat. No. 4,439,443 discloses the use of α-mercapto-β-furylacrylic acids, α-mercapto-β-thienylacrylic acids, α-mercapto-β-pyrrylacrylic acids, or a corresponding disulfide in reducing local hemorrhage and tissue necrosis from the bite or sting of a venomous animal.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

The present invention provides a method for treating a venomous sting or bite by administering to a subject suffering from such a venomous bite or sting an alkaline sodium silicate composition having the empirical formula Na_{8.2}Si_{4.4}H_{9.7}O_{17.6}. This method preferably also reduces local hemorrhage and tissue necrosis at or near the site of the venomous bite or sting.

The present invention also provides a method for inhibiting the toxic effects of venom using the alkaline sodium silicate composition.

### BRIEF DESCRIPTION OF THE DRAWINGS

FIG. 1 is a schematic flow diagram of the process for making the composition.

FIG. 2 is the FTIR spectrum of the alkaline sodium silicate complex composition (SSC).

FIG. 3 is the ¹H-MAS-NMR spectrum of the modified sodium silicate product (SSC).

FIG. 4 is a graph showing the effect of SSC concentration on inhibition of snake and insect venom phospholipaseA₂ (PLA₂).

FIG. 5 is a graph showing the effect of SSC concentration on IT₅₀ inhibition of snake and insect venom PLA₂.

FIG. 6 is a graph showing the effect of SSC concentration on inhibition of snake and insect venom protease.

FIG. 7 is a graph showing the effect of SSC concentration on IT₅₀ inhibition of snake and insect venom protease.

FIGS. 8A and 8B are graphs showing the inhibition of cell viability on human skin melanoma (SK-MEL-28) (FIG. 8B) and mouse myoblast (C2Cl2) (FIG. 8C) cells after 24-h exposure to different C. atrox venom concentrations. Cell viability was determined by the MTT assay. The average cytotoxicity IC₅₀ values were 0.135 mg/mL for human skin melanoma cells and 0.035 mg/mL for mouse myoblast cells. The vertical bars represent the standard deviation. n=3.

FIGS. 9A and 9B are graphs showing the neutralizing capacity of SSC (LIPH) against three IC₅₀ of C. atrox venom on human skin melanoma (SK-MEL-28) (FIG. 9A) and mouse myoblast (C2Cl2) (FIG. 9B) cells. Cell viability after 24 h treatment with different dilutions of LIPH either alone (left bar in pair of bars for each concentration of LIPH) or against 3 IC₅₀ of C. atrox venom (right bar in pair of bars for each concentration of LIPH) was determined by MTT assay. The percentage of cell viability was calculated in comparison to untreated cells taken as 100%. The vertical bars represent the standard deviation. n=3.

FIGS. 10A and 10B show the efficacy of topical application of LIPH in neutralizing hemorrhagic activity of C. atrox venom. For each group of eight mice, one MHD of C. atrox venom (1.5 μg) was s.c. injected into the right side (R) of each mouse (FIG. 10A). Full strength LIPH (0.1 mL) was topically applied immediately with rubbing for 1 min after s.c. injection of one MHD of C. atrox venom into the right side (R) of each mouse (n=8) (FIG. 10B). Full strength LIPH (0.1 mL) was applied onto the left side (L) of each group of mice by pipetting and rubbing for 1 min. Hemorrhagic spots from a representative mouse in each group are shown.

### DETAILED DESCRIPTION OF THE INVENTION

The present invention is directed to the use of an alkaline sodium silicate containing composition in a method for treating a venomous bite or sting in a subject suffering from such a bite or sting and in a method for inhibiting the toxic effects of venom. This composition is disclosed in US Patent Application Publication 2011/0059189 and contains alkaline sodium silicate complex, also referred to herein as SSC. MS and NMR analysis generated a putative empirical formula of the compound or complex to be Na_{8.2}Si_{4.4}H₉₇O_{17.6}. The formula suggests that alkaline sodium silicate complex (SSC) is not a single compound but a mixture of two different compounds that are in equilibrium with each other. Alkaline sodium silicate complex (SSC) appears to be a mixture of:

Trimeric Sodium Silicate (Na₂SiO₃)₃,

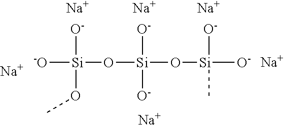

### and Sodium Silicate Pentahydrate (Na₂SiO₃).5H₂O.

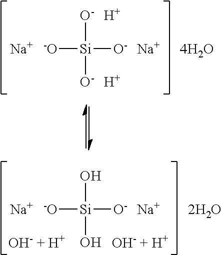

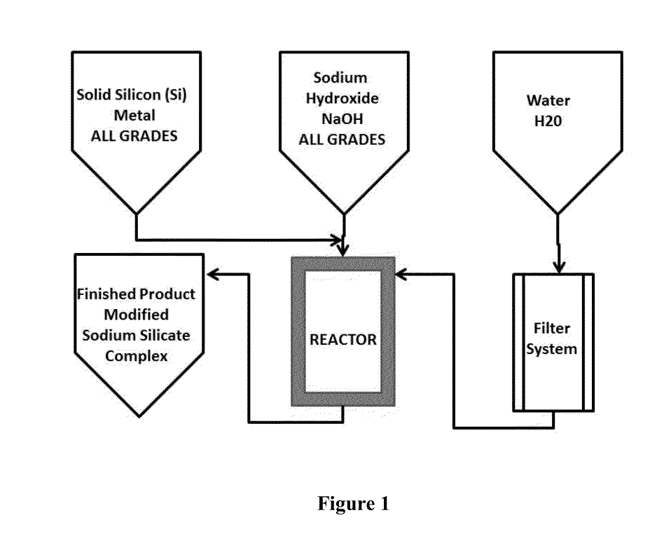

Sodium silicate pentahydrate (Na₂SiO₃).5H₂O appears to exist in equilibrium as two structural forms, with one form containing one ionized water molecule and the other form containing 3 ionized water molecules. To produce alkaline sodium silicate complex (SSC), silicon metal (any grade) is loaded into a reactor. Sodium hydroxide is added along with water. An exothermic reaction occurs. The reaction is allowed to proceed for 4-6 hours, after which the product is collected in a cooling tank. The product is cooled and the obtained liquid product is packaged. FIG. 1 is a schematic flow diagram of the process for making the composition.

In one embodiment, the ingredients for preparing alkaline sodium silicate complex (SSC) are as follows:

about 1-10 parts silicon metal.

about 1-10 parts sodium hydroxide and

about 5-20 parts water.

The silicon metal (1-10 parts) is loaded into the reactor, then 1-10 parts of sodium hydroxide is loaded into the reactor, and 5-20 parts of water is added through a filtration system. An exothermic reaction occurs, which is allowed to continue for about four to six hours. The product is removed from the silicon and reactor, and collected as a liquid in a cooling tank and cooled at ambient room temperature. Water can then be added to reach a specific density of the liquid product. The liquid product can be further filtered. The product obtained is an aqueous solution with an empirical formula of Na_{8.2}Si_{4.4}H_{9.7}O_{17.6}. At this point, the resultant product is in aqueous form and it is ready for packaging and use. It is non-toxic and not corrosive.

In a preferred embodiment for making the SSC product used in the present invention, the silicon used according to the present process is preferably silicon rock of 97-99% purity. Impurities can be less than 1% iron and less than 1% aluminum. The sodium hydroxide solution can have a specific gravity of from 1.11 to 1.53 and can contain from about 40 to about 50% by weight sodium hydroxide.

In a preferred embodiment, the silicon added to the reactor and used in the process to make alkaline sodium silicate complex (SSC) is in rock form (specific gravity of 2.3) and preferably the amount used is in the range of about 40 to 350 pounds, and more preferably in a range of about 46.7 to 300 pounds.

The amount of water used is in the range of about 5.0 to 35 gallons, and more preferably in a range of about 5.5 to 29.8 gallons, and this water heated to a temperature of about 140-150° F.

The amount of the sodium hydroxide (grade 50) used is in a range of about 1.0 to 15.0 gallons, and more preferably in a range of about 2.05 to 11.18 gallons.

As discussed above, the silicon-based alkaline composition (alkaline sodium silicate complex (SSC)) is not a single compound but an aqueous mixture of the following two compounds in equilibrium with each other:

1. Trimeric Sodium Silicate (Na₂SiO₃)₃

2. Sodium Silicate Pentahydrate (Na₂SiO₃).5H₂O

Preferably, the silicon-based alkaline composition (empirical formula of Na_{8.2}Si_{4.4}H_{9.7}O_{17.6}) has a specific density in the range of 1.24 to 1.26, and more preferably the specific density is 1.25+/−. The composition also has a pH in the range of 13.8 to 14.0, and preferably it is 13.9+/−.

Many of the biological activities of alkaline sodium silicate complex (SSC) could be due to the multiple ionized forms, giving it the ability to accept and donate electrons and participate in important redox reactions in the body to bring about redox homeostasis. The elemental and chemical properties of alkaline sodium silicate complex (SSC) give it unique electrochemical and structural characteristics.

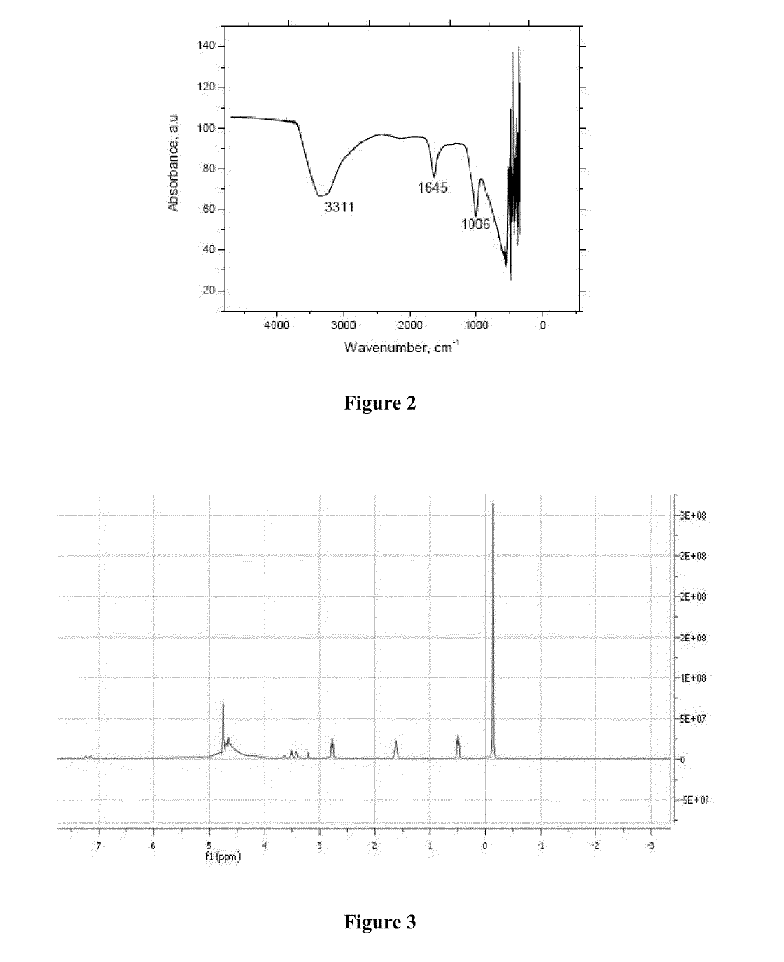

The product used in the method of the present invention is an aqueous solution of Na_{8.2}Si_{4.4}H_{9.7}O_{17.6} (SSC™) with the following properties: a specific density of 1.25+/−, a boiling point of 210° F., a freezing point of 32° F., a pH of 13.9+/−, and solubility in water:miscible 100%. Nuclear magnetic resonance (NMR) and infrared spectroscopy (IR) were performed on the on this product. FIGS. 2 and 3 show the results of IR and NMR studies, respectively. For NMR, an Magic Angle Spinning (MAS) technique of high resolution was used in the ¹H nuclei; meanwhile, for the IR study, Fourier Transform (FT) method was performed to analyze the spectra.

The target solution was analyzed by an IR spectrometer operated in transition mode. FIG. 2 shows three well-resolved vibration signals, the first one at 3311 cm⁻¹ normally attributed to the water molecule in the sample according to discrimination rules followed. The next signals are at 1645 and 1006 cm⁻¹ and are not strain-forward and can be assigned because they can be due to the hydroxyl compounds based on Na and Si or due to the vibration of the Na and Si ions, respectively. These signals can be due to the free OH group. The results shown in FIG. 2 suggests that the only functional groups present in this sample are water and OH groups.

More analysis was carried out using ¹H MAS-NMR to complement the above results. By ¹H MAS-NMR spectroscopy, it was possible to confirm that, in the sample, there are no functional groups other than OH groups. FIG. 3 shows the main resonance of the proton at ˜0 ppm is attributed to water. On the other hand, the tiny peaks observed upwards of 7 ppm can be related to the resonance of the hydroxyl group attached to the inorganic components, Na and/or Si.

²³Na and ²⁹Si NMR experiments can be done to further study the nature and number of substitutes present in the Na and Si shell. Also, the crystalline structure of the sample can be investigated using X-ray diffraction (XRD).

It is common that the victim of a venomous bite or sting does not see (or is unable to identify the species of) the venomous animal that caused the bite or sting. Thus, the lack of reliable species identification, particularly in emergency situations, taken together with the cost of raising antivenom, makes it preferable that the antivenoms used in treatment not be limited in their reactivity to a single species. Polyvalent antivenom may be raised against some or most of the venoms found in any particular geographic area so that in the case of an emergency it can be given without the specific venomous species being reliably identified beforehand. The present methods are distinguished from the prior art in that they use a cost effective alkaline composition containing SSC as a non-antibody based antivenom that, based on the experimental results presented in the Examples hereinbelow, is believed to be universal (or nearly so) against most venoms, regardless of geographical location. Accordingly, the alkaline composition containing SSC is used in the present invention as a polyvalent inhibitor or neutralizer of animal venom.

The term “venom” is intended to encompass any poisonous substance which is parenterally transmitted, that is subcutaneously or intramuscularly transmitted, by the bite or sting of a venomous animal into a mammal and which contains various toxins such as, but not limited to, hemotoxins, hemagglutinins, neurotoxins, leukotoxins, and endotheliatoxins.

The term “venomous animals” is taken to mean venomous members of the Animal kingdom, as are well known in the art and of which representative members are disclosed in the Description of the Related Arts section. Non-limiting examples of venomous animals whose bite or sting transmit venom to a mammal victim include reptiles such as Gila monsters and snakes; arthropods such as spiders (e.g., Black Widow and Brown Recluse spiders) and scorpions; jellyfish, etc. Non-limiting examples of venomous snakes such as pit vipers including Agkistrodon spp., Bothrops spp., Crotalus spp., Trimeresurus spp., Lachesis mutus, and Sistrurus spp. and vipers including Bitis spp., Causus spp., Cerastes spp., Echis carinatus, Pseudocerasters persicus and Vipera spp. Preferably, the venom is from the bite of a venomous snake selected from the group consisting of Crotalus atrox, Agkistrodon contortrix contortrix, and Agkistrodon piscivorus leucostoma.

The term “subject” or “patient” who is suffering from the bite or sting of a venomous animal is a mammal, preferably humans, and includes household pets and livestock, including but not limited to dogs, cats, sheep, horses, cows, goats, and pigs.

In the present method for treating a venomous bite or sting in a subject suffering from such a bite or sting, local hemorrhage and tissue necrosis is preferably reduced at or near the site of the bite or sting. Preferably, such local hemorrhage and tissue necrosis is reduced by topically administering the alkaline sodium silicate containing composition to the skin at or near the site of the bite or sting.

In the present method for inhibiting the toxic effects of venom, an effective amount of an alkaline sodium silicate composition, which comprises the empirical formula Na_{8.2}Si_{4.4}H_{9.7}O_{17.6}, is caused to come into contact with venom to inhibit the toxic effects thereof.

Compositions used within the scope of the present invention include all compositions wherein the SSC is contained in an amount effective to achieve its intended purpose. The term “effective amount” as used herein means an amount effective, at dosages and for periods of time necessary to achieve the desired result. While individual needs vary, determination of optimal ranges of effective amounts of each compound is within the skill of the art. Typical dosages comprise 0.01 to 100 mg/kg body weight. The preferred dosages comprise 0.1 to 100 mg/kg body weight. The most preferred dosages comprise 1 to 50 mg/kg body weight.

Compositions for administering SSC preferably contain, in addition to the SSC, suitable pharmaceutically acceptable carriers comprising excipients and auxiliaries which facilitate processing of the SSC into preparations which can be used pharmaceutically. Preferably, the preparations contain from about 0.01 to about 99 percent by weight, more preferably from about 20 to 75 percent by weight, and most preferably from about 40 to 60 percent by weight SSC together with the excipient.

The pharmaceutically acceptable carriers include vehicles, adjutants, excipients, or diluents that are well known to those skilled in the art and which are readily available. It is preferred that the pharmaceutically acceptable carrier be one which is chemically inert to the SSC and which has no detrimental side effects or toxicity under the conditions of use.

There is a wide variety of suitable formulations of the compositions containing SSC. Formulations can be prepared for topical, oral, aerosol, parenteral, subcutaneous, intravenous, submucosal transdermal, intra arterial, intramuscular, intra peritoneal, and intra tracheal administration. Topical and oral administration are preferred.

Thus, the SSC can be formulated into liquids, creams, gels, pastes, foam, or spray formulations containing, in addition to the active SSC ingredient, such carriers as are known in the art to be appropriate for topical administration. For topical administration, it is preferred that the liquid concentrated SSC product, such as obtained from the process described in Example 1 hereinbelow, be used at full strength (undiluted) or diluted 1:1 with water (50% concentrated liquid SSC product) for application to the subject.

Formulations suitable for oral administration, including submucosal and transbuccal, can consist of liquid solutions such as effective amounts of the SSC diluted in diluents such as water, saline, or orange juice; capsules, tablets, sachets, lozenges, and troches, each containing a predetermined amount of the active ingredient as solids or granules; powders, suspensions in an appropriate liquid; and suitable emulsions. Liquid formulations may include diluents such as water and alcohols, e.g., ethanol, benzyl alcohol, and the polyethylene alcohols, either with or without the addition of a pharmaceutically acceptable surfactant, suspending agents, or emulsifying agents. For oral administration, it is preferred that the liquid concentrated SSC product, such as obtained from the process described in Example 1 hereinbelow, be diluted 1:1 with water (2% concentrated liquid SSC product) for oral administration (by drinking) of the diluted liquid SSC composition. Capsule forms can be of the ordinary hard- or soft-shelled gelatin type containing, for example, surfactants, lubricant, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscaramellose sodium, talc, magnesium stearate, calcium stearate, zinc stearate, stearic acid, and other preservatives, flavoring agents, and pharmaceutically acceptable disintegrating agents, moistening agents preservatives flavoring agents, and pharmacologically compatible carriers. Lozenge forms can comprise SSC in a carrier, usually sucrose and acacia or tragacanth, as well as pastilles comprising SSC in an inert base such as gelatin or glycerin, or sucrose and acacia. Emulsions and the like can contain, in addition to SSC, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The SSC can be administered in a physiologically acceptable diluent in a pharmaceutical carriers, such as a sterile liquid or mixture of liquids, including water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,3-dioxolane-4-methanol, ethers such as poly(ethylene glycol) 400, suspending agent, such as carbomers, methylcellulose, hydroxypropylmethylcellulose, or carboxymethylcellulose, or emulsifying agents and other pharmaceutical adjuvants.

An acute oral toxicity study of the diluted liquid SSC (obtained from the process described in Example 1 by diluting to 2%) was conducted in rats and a skin corrosion/dermal irritation study of the concentrated liquid SSC (obtained from the process described in Example 1 and undiluted) was conducted in rabbits. No clinical signs of toxicity were observed in the rats and no skin corrosion or other signs of skin irritation were observed in the rabbits.

The amount of SSC to be administered to any given patient may be determined empirically, and will differ depending upon the condition of the patients.

Having now generally described the invention, the same will be more readily understood through reference to the following examples which are provided by way of illustration and are not intended to be limiting of the present invention.

### Example 1

### Production of Alkaline Sodium Silicate Complex (SSC)

The following describes a representative, but preferred, method for making SSC,

To make 10 gallons of SSC at 1.25 specific gravity, the following ingredients were used:

| | |
|---|---|
| Initial amount of silicon rock | 46.7 pounds |
| to start the reaction | |
| Water at 150° F. | 5.5 gallons |
| Sodium hydroxide at 50% | 2.05 gallons |

In the reaction process for the first batch, the silicon rock was introduced into a 30 gallon reactor. Note: after the initial reaction, the amount of silicon rock that will be needed to start the reaction for a subsequent second batch and every other thereafter will be only 7.85 pounds.

Second, approximately half of the total volume of the heated water was added to the reactor.

Third, sodium hydroxide was added, while continuing to add the water.

Fourth, the remaining water was added.

Fifth, after all components are added to the reactor, an exothermic reaction occurred for 4 to 6 hours (for the first time batch) (Note: for subsequent batches, the reaction time required will be less). To determine if the reaction is finished, there should be very little reaction bubbles on top of the liquid in the reactor; instead, there are mostly large bubbles and no vapor coming out.

Then, the reaction product was emptied into a tank. This product had very high (like molasses) inconsistency with a temperature of approximately 195 to 200° F. After the reaction product was removed from the reactor, water was sprayed over the remaining silicon rocks in the reactor to wash the rocks, and this washed liquid was emptied into the same tank.

The reaction product was allowed to cool at ambient room temperature. After the reaction product had completely cooled, water was added (while continuously mixing) until a specific gravity (weight in grams of liquid divided by volume in milliliters) of 1.25 was reached. For equal specific gravity, the liquid was allowed to settle for approximately 3 to 4 hours to drop all sediment (black inert material). The liquid was then pumped through a fine filter (1 to 3 microns) and into a storage tank. At this point, the resultant liquid product is ready for packaging into containers and ready to be used. It is non-toxic and not corrosive.

The product obtained was an aqueous solution of Na_{8.2}Si_{4.4}H_{9.7}O_{17.6} with the following properties: a specific density of 1.25+/−, a boiling point of 210° F., a freezing point of 32° F., a pH of 13.9+/−, and solubility in water:miscible 100%.

Nuclear magnetic resonance (NMR) and infrared spectroscopy (IR) were performed on the resultant product. FIGS. 2 and 3 show the results of IR and NMR studies, respectively.

### Example 2

### Effect of SSC on Three Different Types of Snake Venom Using Two Enzymatic Assays

Three snake venoms (Crotalus atrox, Western Diamondback; Agkistrodon contortrix contortrix, Southern Copperhead; and Agkistrodon piscivorus leucostoma, Western Cottonmouth) studied in this Example have numerous proteolytic enzymes in their venoms. Both gelatinase and hide powder azure assays are common methods used in screening venoms for proteolytic activities (Huang and Pérez, 1980; Rinderknecht et al., 1968). This study was designed to measure neutralization of gelatinase and hide powder azure activities in three snake venoms with SSC.

### Methods

### Antigelatinase Assay

A method modified from Sánchez et al. (2003) was used to test the antigelatinase activity of SSC. A 4 mg/mL amount of venoms of C. atrox, A. contortrix contortrix, and A. piscivorus leucostoma was pre-mixed with an equal volume of various dilutions of SSC in normal saline solution (sodium chloride, 0.85% (w/v)). The mixtures were then incubated for 30 min at 37° C. Twenty microliters of each of the mixtures was placed in separate locations on a Kodak X-OMAT scientific imaging film with gelatin coating. The inhibition of hydrolysis of gelatin on the X-ray film was determined by washing the film with tap water after a 4-hr incubation at 37° C. in a moist incubator. The antigelatinase dose is defined as the reciprocal dilution of SSC inhibiting the clearance of the gelatin on the X-ray film. The controls included venom alone (venom control), saline alone (saline control), and undiluted SSC alone (SSC control). This assay was repeated two times.

### Antihide Powder Azure Assay

A modified method of Sánchez et al. (2003) was used to test antihide powder azure activity. Hide powder azure was ground with a mortar and pestle to obtain uniform particles before adding diluent. In separate test tubes, 4 mg/mL of each venom (C. atrox, A. contortrix contortrix, and A. piscivorus leucostoma) was pre-mixed with an equal volume of the undiluted or diluted SSC in normal saline solution (sodium chloride, 0.85% (w/v)). The mixtures were then incubated for 30 min at 37° C. A 1.6 mg of finely ground hide powder azure was mixed with 0.48 mL of normal saline. Twenty microliters of each of the mixtures was then added to the vial. Each vial was incubated for 1 hr at 37° C. and the absorbance measured at 595 nm. The percent inhibition of proteolytic activity is calculated by the following equation: ((C−E/C)×100, where C is the absorbance for the venom control, and E is the absorbance for the experimental fraction. The saline control was hide powder azure incubated with 20 μL of normal saline. The SSC control was hide powder azure incubated with 20 μL of undiluted SSC. The venom control was the hide powder azure incubated with venom only (no SSC). This assay was repeated three times.

### Results

All venoms (40 μg) tested contained gelatinase and hide powder azure activities. The highest dilution of SSC that neutralized gelatinase activity of the venoms of C. atrox, A. contortrix contortrix, and A. piscivorus leucostoma was 1:25, 1:8, and 1:16, respectively (Table 3 below). The SSC was able to inhibit hide powder azure activity of all three venoms (Table 4 below). The highest percent inhibition of SSC that was the most effective in neutralizing hide powder azure activity of the venoms of C. atrox, A. contortrix contortrix, and A. piscivorus leucostoma was 1:8, 1:8, and 1:4, respectively.

TABLE 3
The neutralization of three venoms (40 μg) by SSC using
the antigelatinase assay

| Snake venom | SSC dilution | Antigelatinase activity |
|---|---|---|
| Crotalus atrox | undiluted | + |
| | 1:2 | + |
| | 1:4 | + |
| | 1:8 | + |
| | 1:16 | + |
| | 1:25 | + |
| | 1:32 | − |
| | 1:64 | − |
| | 1:125 | − |
| | 1:2, pH to 7.0* | − |
| Agkistrodon | undiluted | + |
| contortrix contortrix | 1:2 | + |
| | 1:4 | + |
| | 1:8 | + |
| | 1:16 | − |
| | 1:25 | − |
| | 1:32 | − |
| | 1:64 | − |
| | 1:125 | − |
| | 1:2, pH 7.0* | − |
| Agkistrodon | undiluted | + |
| piscivorus | 1:2 | + |
| leucostoma | 1:4 | + |
| | 1:8 | + |
| | 1:16 | + |
| | 1:25 | − |
| | 1:32 | − |
| | 1:64 | − |
| | 1:125 | − |
| | 1:2, pH 7.0* | − |

n = 2.
“+” represents detect antigelatinase activity.
“−” indicates the absence of antigelatinase activity.

Bold numbers in Table 3 indicate the highest dilution of SSC that neutralized gelatinase activity. An asterisk (*) in Table 3 indicates the 1:2 dilution of SSC adjusted to a pH of 7.0 using hydrochloric acid and did not neutralize gelatinase activity. Other dilutions of SSC (1:8, 1:16, 1:25, and 1:32) at pH 7.0 were tested and were incapable of neutralizing the gelatinase activity of all the venoms (data not shown).

TABLE 4
Inhibition of hide powder azure activity by SSC

| | % Inhibition | | |
|---|---|---|---|
| SSC dilution | C.atrox | A.c.contortrix | A.p.leucostoma |
| undiluted | 83 | 77 | 71 |
| 1:2 | 83 | 80 | 82 |
| 1:4 | 81 | 83 | 86 |
| 1:8 | 89 | 84 | 73 |
| 1:16 | 88 | 67 | 74 |
| 1:25 | 82 | 44 | 69 |
| 1:32 | 71 | 0 | 22 |
| 1:64 | 0 | 0 | 0 |
| 1:2, pH 7.0* | 0 | 0 | 0 |

n = 3.

Bold numbers in Table 4 indicate the highest percent inhibition of SSC that was the most effective in neutralizing hide powder azure activity of venoms. An asterisk (*) in Table 4 indicates the 1:2 dilution of SSC adjusted to a pH of 7.0 using hydrochloric acid did not inhibit hide powder azure activity. Other dilutions of SSC (1:8, 1:16, 1:25, and 1:32) at pH 7.0 were tested and had no significant antihide powder azure activity against all three venoms (data not shown).

### Discussion

The gelatinase and hide powder azure assays are fast and easy techniques and are used to test for proteolytic activities in snake venoms. The neutralization of three venoms by the SSC can be measured by these two enzymatic assays. The SSC neutralized the gelatinase activity of all three venoms (Table 3) and also inhibited the hide powder azure activity of all venoms as well (Table 4). Interestingly, only SSC (without venom) partially hydrolyzed hide powder azure resulting in a release of dye from hide powder azure. Thus, the percent inhibitions of undiluted and diluted SSC (1:2 and 1:4) were lower than that of SSC at the higher dilutions.

In most enzyme reactions, high pH reduces enzyme activity. The optimal pH of most enzymes is from 6-8. The pH value of SSC measured using a pH meter is around pH 14, which is a strong base and could denature snake venom enzymes. Other strong basic solutions including sodium hydroxide (pH 14), potassium hydroxide (pH 14), and Clorox (pH 12.50) were used as high pH controls. However, these basic solutions are not good controls since they are corrosive and removed the gelatin on the X-ray film (data not shown). The pH values of SSC at various dilutions (1:2, 1:8, 1:16, 1:25, and 1:32) were reduced to 7.0 and used as the pH control. At pH 7.0, the antigelatinase and antihide powder azure activities of SSC at various dilutions was lost.

### Conclusion

The SSC was able to block proteolytic activities of C. atrox, A. contortrix contortrix, and A. piscivorus leucostoma venoms using two enzymatic assays, gelatinase and hide powder azure assays at pH of 14. When the pH of SSC was adjusted to pH 7, the blocking of proteolytic activity was lost.

### Example 3

### Effect of SSC on Components of Common Snake and Insect Venoms

### Materials and Methods

### Venoms and Toxins:

Venoms and toxins from snakes, scorpions, spiders, bees and wasps relevant to North America will be purchased from Sigma Aldrich (St. Louis, Mo.) and Fisher Scientific.

### Anti-Phospholipase A2 Activity Assay

Phospholipase A2 (PLA₂) activity was measured using an indirect hemolytic assay on agarose-erythrocyte-egg yolk gel plate to define the minimum indirect hemolytic dose (MIHD). The minimum indirect hemolytic dose (MIHD) of venom/toxin will be the dose that induced hemolysis halo having the diameter of 20 mm after incubation for 20 h at 37° C. SSC at various dilutions were tested against one MIHD of each venom/toxin. Test solutions and venom/toxin, 0.05 ml each, were pre-incubated for 1 h at 37° C. After centrifugation at 10,000×g for 10 min, the supernatant were tested for phospholipase A2 activity. The anti-phospholipase A2 potential of SSC is expressed as percent inhibition of the enzyme activity, in which 100% inhibition should produce no clear zone.

### Anti-Proteolytic Activity Assay

To 100 μl of venom/toxin 900 μl of 0.5% azocasein (Sigma Chemical Co., St. Louis, Mo.) prepared in 50 mM Tris buffer containing 2 mM CaCl₂ were added. Samples were incubated at 37° C. for 30 min. After this, 15% TCA (100 ul) were added to stop the reaction. The samples were then centrifuged (7,100 g, 10 min). The supernatants were transferred to separate tubes, and absorbance was measured at 440 nm. Activity was calculated as absorbance of sample at 440 nm divided by the optical density of culture at 650 nm.

### Results

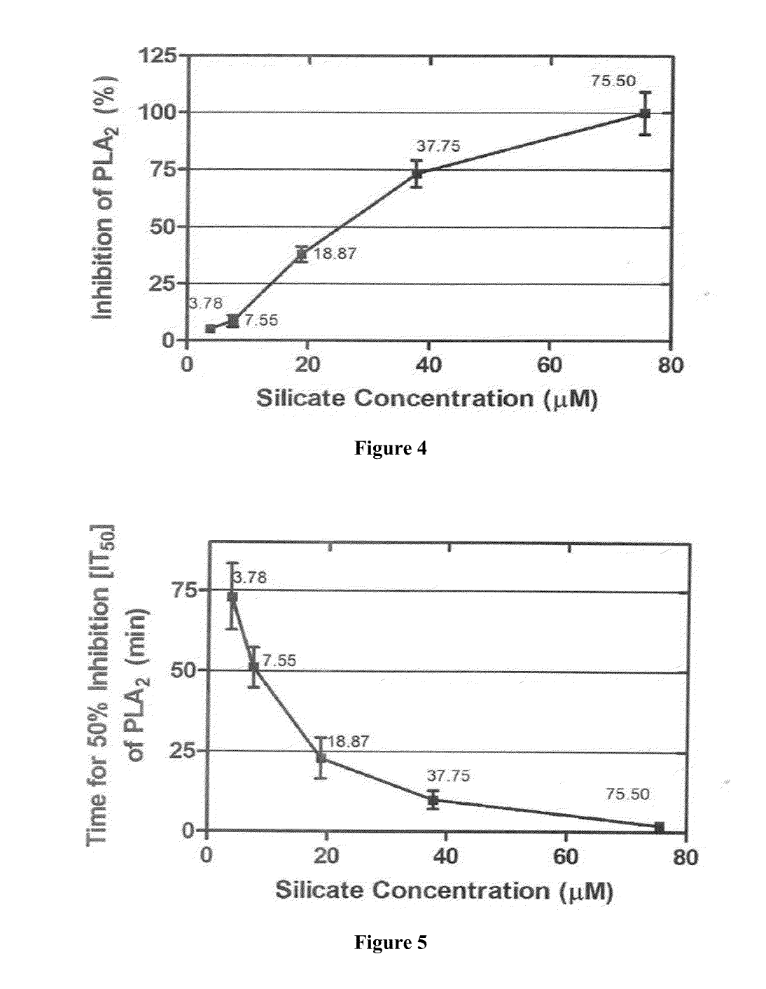

The effect of SSC concentration on inhibition of snake and insect venom PLA₂ activity (responsible for pain, inflammation and neurotoxicity) is shown in FIGS. 4 and 5. In FIG. 4, the activity of PLA₂ is shown to be reduced 75% at a SSC concentration of 37.75 μM and completely (100%) inhibited at a SSC concentration of 75.5 μM. As for FIG. 5, the activity of PLA₂ is shown to be reduced by 50% in 10 minutes at a SSC concentration of 37.75 μM, whereas it was reduced by 50% in 2 minutes at a SSC concentration of 75.5 μM. Accordingly, at a SSC concentration of 37.75-75.5 μM, SSC inhibited the activity of PLA₂ by 75-100% in less than 10 minutes.

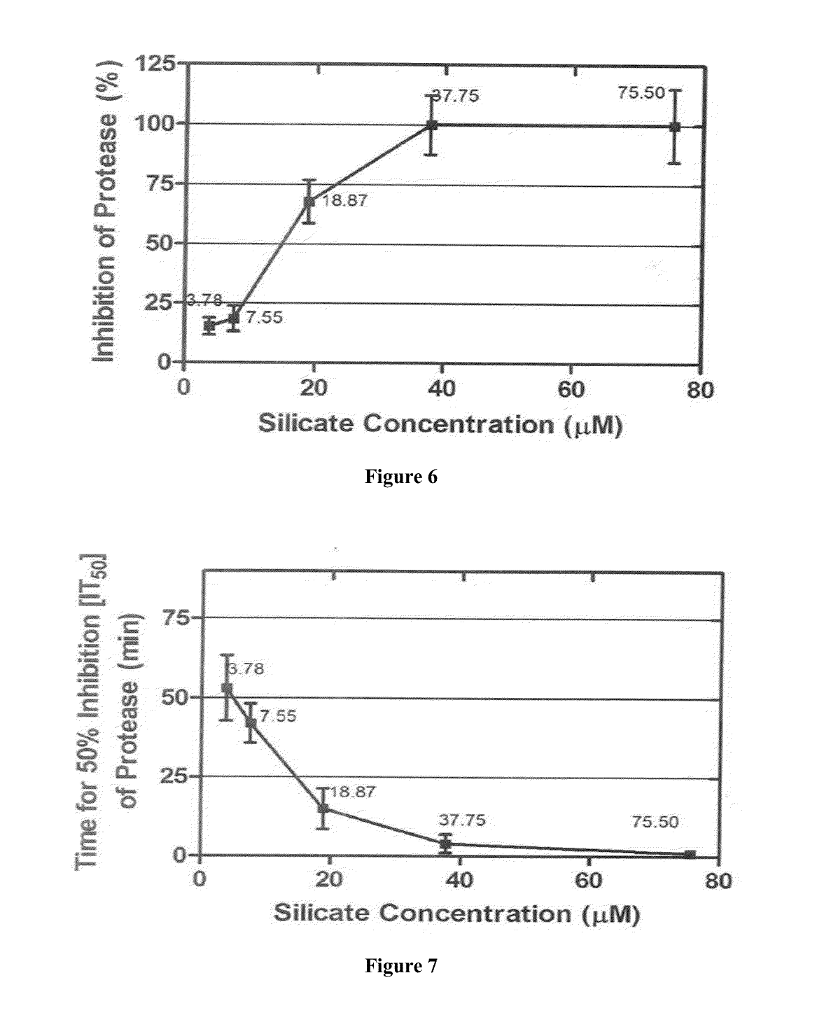

Similarly, the effect of SSC concentration on inhibition of snake and insect venom protease activity (responsible for tissue damage and resulting pain, necrosis and scarring) is shown in FIGS. 6 and 7. In FIG. 6, protease activity is shown to be reduced by 68% at a SSC concentration of 18.87 μM and completely (100%) inhibited at a SSC concentration of 37.75 μM. In FIG. 5, the protease activity is shown to be reduced by 50% in 4 minutes, whereas it was reduced by 50% in 1 minute at a SSC concentration of 75.5 μM. Accordingly, at a concentration of 18.87-37.75 μM, SSC inhibited the activity of protease by 68-100% in less than 5 minutes.

These results show that SSC is effective in reducing the damaging effects of toxins and venoms with the dosage and time needed for inhibition being very low.

### Example 4

Venomous snakebites, although uncommon, are a potentially deadly emergency in the United States. The most acceptable treatment of systemic snake envenomation is antivenom. However, the efficacy of antivenom administration against local symptoms may be poor due to the rapid development of damage at the bite area. Snake proteases, serine proteases, and metalloproteinases, are responsible for the development of local tissue damage and spreading venom components into the circulatory system. Therefore, the neutralization of snake proteases in an early phase of envenomation is needed to prevent local damage and systemic effects. SSC, which is also referred to in the study in this Example as LIPH, at a low concentration is a noncorrosive solution that can denature snake protease enzymes. In this study, LIPH was used to neutralize snake venom using six different assays, which include both in vitro and in vivo assays. Two enzymatic assays, antigelatinase and antihide powder azure assays, were used to measure proteolytic activities of three snake venoms, Crotalus atrox, Agkistrodon contortrix contortrix, and Agkistrodon piscivorus leucostoma. Cell culture assay was used to measure the neutralization of cytotoxicity of C. atrox venom with LIPH. Two different antihemorrhagic assays were used to measure the ability of LIPH in preventing hemorrhage in mice. An antilethality (ED₅₀) assay was used to test the efficacy of LIPH in preventing death in mice.

Abbreviations used in this example are: C2Cl2, mouse myoblast; DMEM, Dulbecco's Modified Eagle's Medium; DMSO, dimethyl sulfoxide; EMEM, Eagle's minimum essential medium; FBS, fetal bovine serum; IC₅₀, 50% inhibitory concentration; LD₅₀, lethal dose; MHD, minimal hemorrhagic dose; MTT, 3-(4,5-Dimethylthiazol-2-yl)-2,5-diphenyltetrazolium bromide; SK-MEL-28, human skin melanoma.

### Materials and Methods

### Venom

Crude lyophilized venom of Western Diamondback Rattlesnake (Crotalus atrox), Southern Copperhead (Agkistrodon contortrix contortrix), and Western Cottonmouth (Agkistrodon piscivorus leucostoma) were purchased from National Natural Toxins Research Center (NNTRC) at Texas A&M University-Kingsville. Lyophilized crude venoms were pooled from the same species covering the entire range of each venom.

### Animal

All animal studies and procedures were approved by the Institutional Animal Care and Use Committee (IACUC) and were conducted in accordance with the National Institute of Health Animal Care Guidelines. Female BALB/c mice (18-20 g) were used for all animal studies.

### pH of the LIPH Solution

The pH of the LIPH solution was measured with a pH meter.

### Antigelatinase Assay

A method modified from Sánchez et al. (2003) was used to test the antigelatinase activity of the LIPH. 4 mg/mL of venoms from C. atrox, A. contortrix contortrix, and A. piscivorus leucostoma were pre-mixed with an equal volume of the undiluted or diluted LIPH in normal saline solution (sodium chloride, 0.85% (w/v)). The mixtures were then incubated for 30 min at 37° C. Twenty microliters of each of the mixtures were placed in separate locations on a Kodak X-OMAT scientific imaging film with gelatin coating. The inhibition of the hydrolysis of gelatin on the X-ray film was determined by washing the film with tap water after a 4-hr incubation at 37° C. in a moist incubator. The antigelatinase dose was defined as the reciprocal dilution of LIPH inhibiting the clearance of the gelatin on the X-ray film. The controls included venom alone (venom control), saline alone (saline control), and undiluted LIPH alone (LIPH control). This assay was repeated two times.

### Antihide Powder Azure Assay

A modified method of Sánchez et al. (2003) was used to test antihide powder azure activity. Hide powder azure was ground with a mortar and pestle to obtain uniform particles before adding diluent. In separate test tubes, 4 mg/mL of each venom (C. atrox, A. contortrix contortrix, and A. piscivorus leucostoma) were pre-mixed with an equal volume of the undiluted or diluted LIPH in normal saline solution (Sodium Chloride, 0.85% (w/v)). The mixtures were then incubated for 30 min at 37° C. A 1.6 mg of finely ground hide powder azure was mixed with 0.48 mL of normal saline. Twenty microliters of each of the mixtures were then added to the vial. Each vial was incubated for 1 hr at 37° C. and the absorbance measured at 595 nm. The percent inhibition of proteolytic activity is calculated by the following equation: [(C−E/C]×100, where C is the absorbance for the venom control, and E is the absorbance for the experimental fraction. The saline control was hide powder azure incubated with 20 μL of normal saline. The LIPH control was hide powder azure incubated with 20 μL of undiluted LIPH. The venom control was the hide powder azure incubated with venom only (no LIPH). This assay was repeated three times.

### Human Melanoma (SK-MEL-28) and Mouse Myoblast (C2Cl2) Cell Lines

The human melanoma (SK-MEL-28) and mouse myoblast (C2Cl2) cell lines were obtained from American Type Tissue Culture Collection (ATCC). The human melanoma cells were maintained with Eagle's minimum essential medium (EMEM) containing 10% fetal bovine serum (FBS), 100 U/mL penicillin, and 100 μg/mL streptomycin. The mouse myoblast cells were maintained in Dulbecco's Modified Eagle's Medium (DMEM) containing 10% FBS, 50 U/mL penicillin, and 50 μg/mL streptomycin. The cultured medium was replaced daily. Cells were incubated at 37° C. in a 5% CO₂ humidified incubator.

Cytotoxicity Assay of C. atrox Venom on Cell Lines

The cytotoxic activity of C. atrox venom was performed on human melanoma and mouse myoblast cell lines using MTT (3-(4,5-Dimethylthiazol-2-yl)-2,5-diphenyltetrazolium bromide) assay. Cells were cultured on a 96-well flat-bottom microtiter plate at 10⁵ cells/well for human melanoma cells and 1.5×10⁵ cells/well for mouse myoblast cells in triplicate and incubated at 37° C. in 5% CO₂ for 24 h. Twenty microliters of C. atrox venom at various concentrations in sterile 0.85% saline were added to cell suspension at 37° C. for 24 h. Then, 12 μL of MTT (5 mg/mL) were added to each well. After incubation for 4 h at 37° C., the culture medium was aspirated and 100 μL of dimethyl sulfoxide (DMSO) were added to lyse the cells. The absorbance at 570 nm was read using a Beckman Coulter™ model AD 340 reader. Cells treated with 0.85% saline were used as the control. The concentration of cytotoxicity was calculated using the following equation: [(C-E/C)]×100, where C is the absorbance of the control, and E is the absorbance of the experimental fraction. The 50% inhibitory concentration (IC₅₀) is defined as the venom concentration that inhibits cell viability by 50%. The values of the concentrations of cell viability inhibition were plotted against venom concentrations, and the IC₅₀ were determined. The IC₅₀ values are average determinations from two independent experiments.

Neutralizing Capacity of LIPH Against the Cytotoxicity of C. atrox Venom on Cell Lines

The efficacy of LIPH against C. atrox venom was performed on human melanoma and mouse myoblast cell lines. Cells were cultured on a 96-well flat-bottom microtiter plate at 10⁵ cells/well for human melanoma cells and 1.5×10⁵ cells/well for mouse myoblast cells in triplicate and incubated at 37° C. in 5% CO₂ for 24 h. In separate test tubes, the crude venom containing three IC₅₀ were pre-mixed with an equal volume of the undiluted or diluted LIPH in 0.85% saline and incubated for 30 min at 37° C. Twenty microliters of the sample mixture were added to cell suspension at 37° C. for 24 h. Then, 12 μL of MTT (5 mg/mL) were added to each well. After incubation for 4 h at 37° C., the culture medium was aspirated and 150 μL of DMSO were added to lyse the cells. The absorbance at 570 nm was read using a Beckman Coulter™ model AD 340 reader. The control groups included venom alone (C. atrox control), saline alone (saline control), and LIPH alone (LIPH control). Only the culture medium (no cell) with the sample was used as the blank. Doxorubicin (177 μM), an anticancer drug, was used as the positive control. The percentage of cell viability was calculated using the following equation: [(Aₑₓₚ−A_{b})/(A_{c}−A_{b})]×100, where Aₛₐₘₚₗₑ is the absorbance of the experimental fraction, A_{b} is the absorbance of the blank, and A_{c} is the absorbance of the saline control. Experiments were performed in triplicate.

### The Inhibitory Capacity of LIPH on Local Hemorrhage

### Hemorrhagic Assay

A hemorrhagic assay was performed to determine the ability of proteases in venom fostered bleeding. A modified method described by Sánchez et al. (2011) was routinely used to determine the minimal hemorrhagic dose (MHD) for the crude venom of C. atrox. A set of dilution was made for the venom sample, of which 0.1 mL of each dilution was injected subcutaneously (s.c.) into the backs of BALB/c mice (n=8) to test for activity. After 24 h, the mice were sacrificed. The hemorrhagic spots were measured (mm) and the MHD determined. The MHD is defined as the amount of venom protein, which causes a 10 mm hemorrhagic spot.

### Antihemorrhagic Assay

The antihemorhagic assay was performed to determine the efficacy of LIPH that neutralized two MHD of C. atrox venom in mice. A modified method used by Sánchez et al. (2003) was followed. In separate test tubes, crude venom containing two MHD was pre-mixed with an equal volume of diluted LIPH in 0.85% saline and incubated for 60 min at 25° C. The backs of mice were depilated and 0.1 mL of each mixture was injected s.c. in mice. The control groups included venom alone (venom control), saline alone (saline control), LIPH alone (LIPH control). The percent inhibition of hemorrhagic activity was calculated using the following equation: [(C−E/C)]×100, where C is the hemorrhagic spot in diameter (mm) by 1.5 μg of C. atrox venom, and E is the hemorrhagic spot in diameter (mm) of the experimental fraction.

### Topical Application Assay

The efficacy of topical application of LIPH that neutralized one MHD of C. atrox venom in mice was determined by the following procedure. For each group of eight mice, 0.1 mL of one MHD of venom was injected s.c. into the right side of each mouse. Immediately after venom injection, 0.1 mL of full strength of LIPH was applied by pipetting and rubbing for 1 min on the venom injection site (right side) and the left side as a LIPH control. After 24 h, the mice were sacrificed. Responses were determined by measuring the diameters of the target lesions. The efficacy of the topical application of LIPH was compared with the control groups. The control groups included venom alone (venom control), saline alone (saline control), and LIPH alone (LIPH control).

Effect of LIPH in Neutralizing Lethality Induced by C. atrox Venom

### Lethal Dose (LD₅₀)

The lethal dose (LD₅₀) assay is a common method to determine the toxicity of snake venom. We routinely use a method described by Sánchez et al. (2003) to determine the LD₅₀ for crude venoms. Five groups of eight mice for C. atrox venom were housed in cages and observed throughout the quarantine period and experiments. The previously reported intravenous (i.v.) lethal dose for C. atrox ranged from 4.07-8.42 mg/kg body weight (Tennant and Barlett, 2000). Venom was dissolved in physiological saline at the highest concentration of venom that was used for the injection. The highest concentration was approximately four times higher than the previously reported mean LD₅₀. Two-fold serial dilutions using saline were made to obtain four additional concentrations. All solutions during the experiment were stored at 4° C. and warmed to 37° C. just before being injected into mice. The lethal toxicity was determined by injecting 0.2 mL of venom (at various concentrations) into the tail veins of 18-20 g female BALB/c mice. The injections were administered using a 1 mL syringe fitted with a 30-gauge, 0.5-in. needle. A saline control was used. The endpoint of lethality of the mice was determined after 48 h. The calculations for the LD₅₀ were generated by a program on the NNTRC homepage (ntrc.tamuk.edu/LD50Calculator.xls), which was based on the method developed by Saganuwan (2011).

### Antilethal Dose (ED₅₀)

The antilethal dose assay was performed to determine the effective dose of LIPH that neutralized three LD₅₀ of C. atrox venom in mice. It is a useful preclinical test of in vivo neutralization potency. The procedure used by Sánchez et al. (2003) at the NNTRC was followed. Six groups of eight mice were challenged with a mixture of LIPH containing three LD₅₀ of venom. Various dilutions of LIPH were used. All subsequent dilutions were made with sterile 0.85% saline. Stock venom solutions were freshly prepared at 0° C. before being used. For each group of mice, three venom LD₅₀ were mixed with diluted LIPH in sterile 0.85% saline. The mixtures were incubated at 37° C. for 30 min. Each mouse was injected with 0.2 mL of the mixture freshly mixed and into the tail vein of mice. The mice were observed for 48 h and the survival was recoded. The control groups included venom alone (venom control), saline alone (saline control), LIPH alone (LIPH control).

### Results

### Antigelatinase and Antihide Powder Azure Assays

All venoms (40 μg) contained gelatinase and hide powder azure proteolytic activities. The lowest concentration of LIPH that neutralized gelatinase activity of the venoms of C. atrox, A. contortrix contortrix, and A. piscivorus leucostoma was 4%, 12.5%, and 6.25% (v/v), respectively (Table 5).

TABLE 5
The neutralization of three venoms (40 μg) by LIPH using
the antigelatinase assay.

| Snake venom | LIPH concentration (v/v) | Antigelatinase activity |
|---|---|---|
| Crotalus atrox | 0.8% | − |
| | 1.6% | − |
| | 3% | − |
| | 4% | + |
| | 6.25% | + |
| | 12.5% | + |
| | 25% | + |
| | 50% | + |
| | Full strength | + |
| | 50%, pH to 7.0* | − |
| Agkistrodon | 0.8% | − |
| contortrix contortrix | 1.6% | − |
| | 3% | − |
| | 4% | − |
| | 6.25% | − |
| | 12.5% | + |
| | 25% | + |
| | 50% | + |
| | Full strength | + |
| | 50%, pH to 7.0* | − |
| Agkistrodon | 0.8% | − |
| piscivorus | 1.6% | − |
| leucostoma | 3% | − |
| | 4% | − |
| | 6.25% | + |
| | 12.5% | + |
| | 25% | + |
| | 50% | + |
| | Full strength | + |
| | 50%, pH to 7.0* | − |

n = 2.
“+” represents detected antigelatinase activity.
“−” indicates the absence of antigelatinase activity.
A bold number indicates the highest concentration of LIPH that neutralized gelatinase activity.
An asterisk (*) indicates at 50% (v/v) LIPH adjusted to a pH of 7.0 using hydrochloric acid and did not neutralize gelatinase activity. Other concentrations of LIPH (12.5%, 6.25%, 4%, and 3% (v/v)) at pH 7.0 were tested and were incapable of neutralizing the gelatinase activity of all the venoms (data not shown).

The LIPH was able to inhibit hide powder azure activity of all three venoms (Table 6). The lowest concentration of LIPH that was the most effective in neutralizing hide powder azure activity of the venoms of C. atrox, A. contortrix contortrix, and A. piscivorus leucostoma was 12.5%, 12.5%, 25% (v/v), respectively.

TABLE 6
Inhibition of hide powder azure activity by LIPH.

| | % Inhibition | | |
|---|---|---|---|
| LIPH concentration (v/v) | C.atrox | A.c.contortrix | A.p.leucostoma |
| 1.6% | 0 | 0 | 0 |
| 3% | 71 | 0 | 22 |
| 4% | 82 | 44 | 69 |
| 6.25% | 88 | 67 | 74 |
| 12.5% | 89 | 84 | 73 |
| 25% | 81 | 83 | 86 |
| 50% | 83 | 80 | 82 |
| Full strength | 83 | 77 | 71 |
| 50%, pH to 7.0* | 0 | 0 | 0 |

n = 3.
A bold number indicates the highest percent inhibition of LIPH that was the most effective in neutralizing hide powder azure activity of venoms.
An asterisk (*) indicates at 50% (v/v) LIPH adjusted to a pH of 7.0 using hydrochloric acid did not inhibit hide powder azure activity. Other concentrations of LIPH (12.5%, 6.25%, 4%, and 3% (v/v)) at pH 7.0 were tested and had no significant antihide powder azure activity against all three venoms (data not shown).

Neutralizing Capacity of LIPH Against the Cytotoxicity of C. atrox Venom on Cell Lines

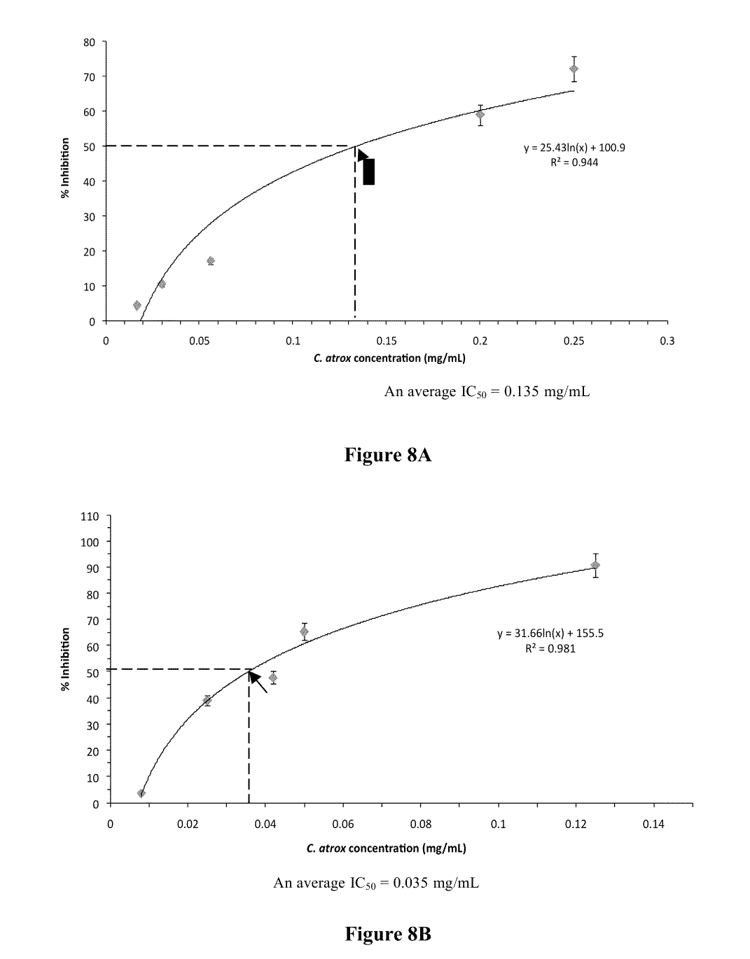

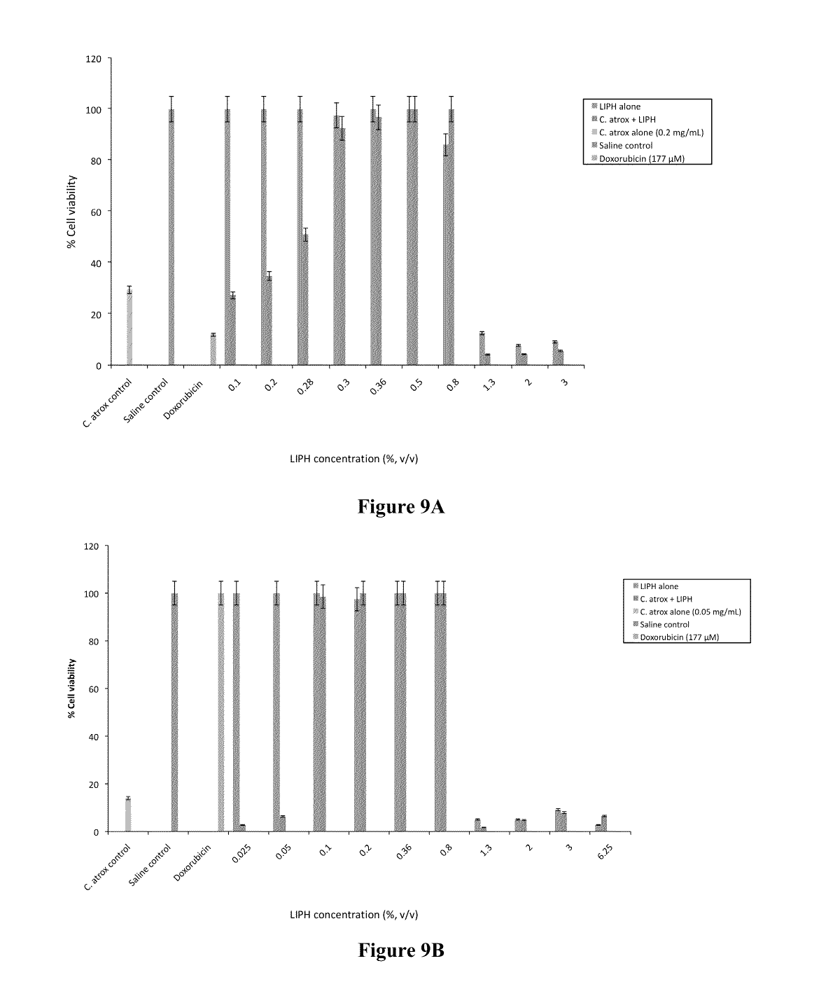

C. atrox venom showed cytotoxic effects with average IC₅₀ values of 0.135 mg/mL for human melanoma cells and 0.035 mg/mL for mouse myoblast cells (FIGS. 8A and 8B). The mouse myoblast cells are more sensitive to C. atrox venom than the human melanoma cells. The IC₅₀s were further used for testing the efficacy of LIPH in neutralizing cytotoxicity of C. atrox venom on two different cultured cells. Full strength LIPH alone prevented cell growth in both cell lines but concentrations of LIPH less than 1.3% (v/v) did not prevent cell growth (FIGS. 9A and 9B). The lowest concentration of LIPH that neutralized three IC₅₀ of C. atrox venom on human skin melanoma and mouse myoblast cells was 0.3% (v/v) with 92% cell survival and 0.1% (v/v) with 99% cell survival, respectively.

### The Inhibitory Capacity of LIPH on Local Hemorrhage

C. atrox venom is the most hemorrhagic venom when compared to A. contortrix contortrix, and A. piscivorus leucostoma (Sanchez et al., 2003). The MHD of C. atrox venom (1.5 μg) was used to test the efficacy LIPH in preventing hemorrhage. The MHD of C. atrox venom was 1.5 μg when injected s.c. in mice. Full strength LIPH alone caused local hemorrhagic after 24 h i.m. injection of LIPH (data not shown). Concentrations of LIPH greater than 0.7% (v/v) also showed hemorrhagic activity after s.c. injection in mice. However, low concentrations of LIPH less than or equal to 0.4% (v/v) did not cause hemorrhage (data not shown). Concentrations of LIPH with no hemorrhage activity (0.4%, 0.3%, 0.25%, and 0.2% (v/v)) were further tested for the neutralization of two MHD of C. atrox venom when injected s.c. in mice. At 0.4% (v/v) LIPH caused the maximum reduction of hemorrhagic activity of C. atrox venom by 29% (Table 7).

TABLE 7
The neutralization of hemorrhagic activity of C. atrox venom
(1.5 μg) by LIPH using the antihemorrhagic assay.

| LIPH concentration (v/v)^{a} | Inhibition of hemorrhagic activity (%) |
|---|---|
| C. atrox venom control | — |
| 0.2% | 8 |
| 0.25% | 26 |
| 0.3% | 28 |
| 0.4% | 29 |

n = 8
An asterisk (*) indicates the minimal hemorrhagic dose (MHD) of C. atrox venom. The MHD is defined as the amount of venom protein, which causes a 10 mm hemorrhagic spot.
A bold number indicates the highest percent inhibition of LIPH that was the most effective in neutralizing hemorrhagic activity of C. atrox enom.
^{a}Two MHD of C. atrox venom were pre-mixed with an equal volume of diluted LIPH in 0.85% saline and incubated for 60 min at 25° C. The backs of mice were depilated and 0.1 mL of each mixture was injected s.c.

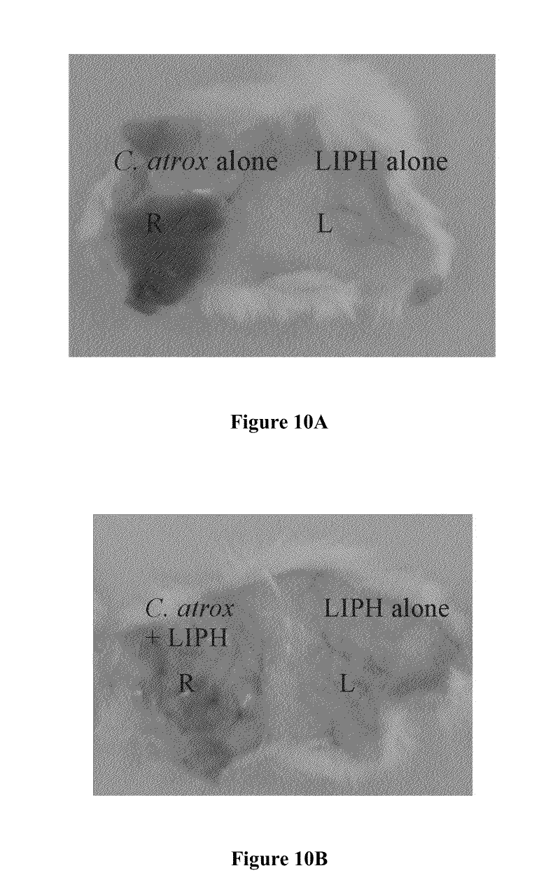

Full strength LIPH did not cause hemorrhage when applied topically. The efficacy of topical application of LIPH against one MHD of C. atrox venom in mice was determined. The topical application of LIPH when rubbed for 1 min neutralized hemorrhagic activity after s.c. injection of one MHD of C. atrox in mice (FIGS. 10A and 10B).

Effect of LIPH in Neutralizing Lethality Induced by C. atrox Venom

The i.v. LD₅₀ of C. atrox venom was 3 mg/kg body weight and three LD₅₀ were used to test efficacy of LIPH in neutralizing venom. The i.v. injections of LIPH greater than 0.8% (v/v) were lethal. However, no mice died after i.v. injection of 0.7% (v/v) LIPH. The effective dose of diluted LIPH that neutralized three LD₅₀ of C. atrox venom in mice was further determined using the antilethal dose assay. At 0.5% (v/v) LIPH completely neutralized three LD₅₀ of C. atrox venom (Table 8).

TABLE 8
The neutralization of lethality of three LD₅₀ of C. atrox venom
by LIPH using antilethal dose assay.

| Sample injection | Died | Lived | Survival rate (%) |
|---|---|---|---|
| 3LD₅₀ of C. atrox + 0.025% (v/v) LIPH | 6 | 2 | 25 |
| 3LD₅₀ of C. atrox + 0.05% (v/v) LIPH | 5 | 3 | 60 |
| 3LD₅₀ of C. atrox + 0.01% (v/v) LIPH | 1 | 7 | 62.5 |
| 3LD₅₀ of C. atrox + 0.2% (v/v) LIPH | 1 | 7 | 62.5 |
| 3LD₅₀ of C. atrox + 0.5% (v/v) LIPH | 0 | 8 | 100 |
| 3LD₅₀ of C. atrox + 1% (v/v) LIPH | 1 | 7 | 87.5 |
| 3LD₅₀ of C. atrox + 1.4% (v/v) LIPH | 3 | 5 | 62.5 |
| C. atrox venom control^{a} | 7 | 1 | 12.5 |

n = 8
^{a}Three LD₅₀ of C. atrox venom were pre-mixed with an equal volume of normal saline and incubated for 30 min at 37° C. Each mouse was injected with 0.2 mL of a mixture into the tail vein.
A bold number indicates the highest concentration of LIPH that completely neutralized three LD₅₀ of C. atrox venom.

### Discussion

Envenomation with Viperidae snake venoms can be a painful and terrifying experience that generally results in edema, necrosis, hemorrhage, coagulopathy and, in some cases, death. The gelatinase and hide powder azure assays are fast and easy techniques and were used to test for proteolytic activities in snake venoms. The neutralization of three venoms by LIPH was measured by enzymatic assays. LIPH neutralized the gelatinase activity of all three venoms (Table 5). LIPH also inhibited the hide powder azure activity of all venoms (Table 6). LIPH without venom partially hydrolyzed hide powder azure resulting in a release of dye; however, lower concentrations of LIPH neutralized enzymatic activity of the venoms.

In most enzyme reactions, a high pH reduces enzyme activity. The optimal pH of most enzymes is from 6-8. The pH value of LIPH measured using our pH meter is around pH 14, which is a strong base and could denature snake venom enzymes. Other strong basic solutions including sodium hydroxide (pH 14), potassium hydroxide (pH 14), and Clorox (pH 12.50) were used as high pH controls. However, these basic solutions are not good controls since they are corrosive and removed the gelatin on the X-ray film (data not shown). The pH values of 3%, 4%, 6.25%, 12.5%, 50% (v/v) LIPH were reduced to 7.0 and used as the pH control. At pH 7.0, the antigelatinase and antihide powder azure activities of LIPH at various dilutions were lost.

The cytotoxic effects induced by C. atrox venom on two different cell lines, human skin melanoma and mouse myoblast cells were also analyzed. Crude venom from pooled C. atrox snakes was dose-dependently cytotoxic for both cell lines. C. atrox venom was about 4 times more toxic on the mouse myoblast cells than the human skin melanoma cell. This is not surprising since the sources and types of cell lines used were different.

In this study, the cytotoxic effects of LIPH on human skin melanoma and mouse myoblast cells were investigated. Concentrations of LIPH greater than or equal to 1.3% (v/v) had a cytotoxic effect on both cell lines, since the LIPH is a strong basic solution and the cultured cells are sensitive to a high pH. However, lower concentrations of LIPH less than or equal to 0.8% (v/v) did not affect cell growth. The neutralizing effect of LIPH against the cytotoxicity of C. atrox venom was maximal at 0.3% (v/v) LIPH for human skin melanoma cells and at 0.1% (v/v) LIPH for mouse myoblast cells. This indicated that the neutralizing capacity of LIPH against the cytotoxicity of C. atrox venom on mouse myoblast cells is more efficient than the human skin melanoma cells.

Any ideal antivenom should protect the most sensitive human from all snake venoms without any side effects. Full strength LIPH had hemorrhagic activity after i.m. injection in mice. High concentrations of LIPH greater than 0.7% (v/v) also caused hemorrhage after s.c. injection. At 0.4% (v/v) LIPH reduced the hemorrhagic activity of C. atrox venom by 29%. However, the neutralization of hemorrhage activity of C. atrox was improved by topical application of full strength LIPH (FIGS. 10A and 10B). Snake venom hemorrhagic metalloproteinases are known to cause local tissue damage (hemorrhage, edema, and necrosis) by degradation of basement membrane and extracellular matrix surrounding capillaries and small vessels. They also play an important role in the development of coagulopathy by causing rapid spreading of procoagulants from the venom injection site into the systemic circulation (Anai et al., 2002; Bjarnason and Fox, 1994; Baramova et al., 1989). It has been reported that the neutralization of venom hemorrhagic metalloproteinases prevents coagulopathy in an animal model (Anai et al., 2002).

LIPH at high concentration was lethal since the LIPH is a strong basic solution (pH 14). Ideally, the pH of the blood should be maintained at 7.4. If the pH drops below 6.8 or rises above 7.8, death may occur. High concentration of LIPH greater than 0.8% (v/v) was lethal to mice but at 0.5% (v/v), LIPH neutralized the lethality induced by three LD₅₀ of C. atrox venom in mice.

### Conclusion

LIPH was used to neutralize snake venom using six different assays including antigelatinase assay, antihide powder azure assay, cell culture assay, two different antihemorrhagic assays, and an antilethality (ED₅₀) assay. LIPH was able to block proteolytic activities of C. atrox, A. contortrix contortrix, and A. piscivorus leucostoma venoms. High concentrations of LIPH were cytotoxic to tissue culture cells, caused hemorrhage in mice, and lethal when injected i.v. However, lower concentrations of LIPH were able to neutralize the cytotoxicity, prevent hemorrhage, and reduce lethality of C. atrox venom. Interestingly, the topical application of full strength of LIPH neutralized and prevented the hemorrhage induced by C. atrox venom. Therefore, topical application is a promising treatment for local tissue damage after snakebite. Topical application of LIPH in the field as a first aid treatment would be especially useful in remote areas far from medical facilities.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### REFERENCES

Anai, K., Sugiki, M., Yoshida, E., Maruyama, M. (2002). Neutralization of a snake venom hemorrhagic metalloproteinase prevents coagulopathy after subcutaneous injection of Bothrops jararaca venom in rats. Toxicon 40, 63-68.
Ayeb et al., In: Handbook of Natural Toxins, Vol. 2, Insect Poisons, Allergens, and Other Invertebrate Venoms, (Anthony T. Tu, Ed.) (Marcel Dekker 1984), Chapter 18 (pp. 607-638)
Baramova, E. N., Shannon, J. D., Bjarnason, J. B., Fox, J. W. (1989). Degradation of extracellular matrix proteins by hemorrhagic metalloproteinases. Arch Biochem Biophys. 275, 63-71.
Baxter et al., Toxicon, 7:195 (1969)
Bjarnason et al., Biochemistry, 17(16):3395 (1978)
Bjarnason, J. B., Fox, J. W. (1994). Hemorrhagic metalloproteinase's from snake venoms. Pharmacol. Ther. 62, 325-372.
Braud, S., Bon, C., Wisner, A. (2000). Snake venom proteins acting on hemostasis. Biochimie 82, 851-859.
Christopher et al., S. Med. J. 79:159 (1986)
Curry et al., J. Toxicol.—Clin. Toxicol. 21417 (1983-1984)
Dart, R. C., McNally, J. (2001). Efficacy, Safety and Use of Snake Antivenoms in the United States, Ann Emerg Med 37, 181-188.
Ellenhorn et al., Medical Toxicology, Ch. 39 (Elsevier Press 1988)
Endean, Toxicon, 25:483 (1987)
Evans, J., Ownby, C. L. (1999). Neutralization of edema, hemorrhage and myonecrosis induced by North American crotalid venoms in simulated first-aid treatments. Toxicon 37, 633-650.
Friederick et al., Biochem. Pharm. 20 1549 (1971)
Gold, B. S., Dart, R. C., Barish, R. A. (2002). Bites of venomous snakes. N Engl J Med 347, 347-356.
Gold, B. S., Barish, R. A., Dart, R. C. (2004). North American snake envenomation: diagnosis, treatment, and management. Emerg Med Clin N Am 22, 423-443.
Gutierrez, J. M., Rucavado, A. (2000). Snake venom metalloproteinases: their role in the pathogenesis of local tissue damage. Biochimie 82, 841-850.
Habermehl, Venomous Animals and Their Toxins (Springer-Verlag, Berlin 1981)
Hassan, In: Handbook of Natural Toxins, Vol. 2, Insect Poisons, Allergens, and Other Invertebrate Venoms, (Anthony T. Tu, Ed.) (Marcel Dekker 1984), Chapter 17 (pp. 577-605)
Huang, S. Y., Pérez, J. C., Comparative study on hemorrhagic and proteolytic activities of snake venoms. Toxicon. 18:421-426 (1980).
Iddon et al., Toxicon 26:167 (1988)
Khunsap, S., Pakmanee, N., Khow, O., Chanhome, L., Sitprija, V., Suntravat, M., Lucena, S. E., Pérez J. C., Sánchez, E. E. (2011). Purificacion of a phospholipase A₂ form Daboia russelii siamensis venom with anticancer effects. J Venom Res, 2, 42-51.
Lumley et al., Med. J. Aust. 148:527 (1988)
Martinez et al., Toxicon, 27:239 (1989)
Olson et al., Toxicon, 22:733 (1984)
Ownby et al., J. Clin. Pharm., 15 419 (1975)
Ownby et al., supra. Holvey, D. N., ed., The Merck Manual of Diagnosis and Therapy, 13th Edition, Merck Sharp and Dohme Research Laboratories, Rahway, N.J., 1972, page 1987.
Ownby, C. L., Colberg, T. R., Odell, G. V. (1986). In vivo ability of antimyotoxin a serum plus polyvalent (Crotalidae) antivenom to neutralize prairie rattlesnake (Crotalus viridis viridis) venom. Toxicon 24, 197-200.
Parrish et al., Clin. Tox. 3:501 (1970).
Parrish, Public Health Rpt. 81:269 (1966)
Perez et al., Toxicon, 22:967 (1984)
Perez, J. C., Sánchez, E. E. (1999). Natural protease inhibitors to hemorrhagins in snake venoms and their potential use in medicine. Toxicon 37, 703-728.
Rinderknecht, H., Geokas, M. C., Silverman, P., Haverback, B. J., A new ultrasensitive method for the determination of proteolytic activity. Clin Chim Acta. 21:197-203 (1968).
Rucavado, A., Escalante, T., Franceschi, A., Chaves, F., Leon, G., Cury, Y., Ovadia, M., Gutierrez, J. M. (2000) Inhibition of local hemorrhage and dermonecrosis induced by Bothrops asper snake venom: effectiveness of early in situ administration of the peptidomimetic metalloproteinase inhibitor batimastat and the chelating agent CaNa2EDTA. Am. J. Trop. Med. Hyg. 63, 313-319.
Russell et al., JAMA 233:341 (1975).
Russell et al., Toxicon 8:63 (1970)
Russell, JAMA 215:1994 (1971)
Russell, F. E., Ruzic, N., Gonzalez, H. (1973). Effectiveness of antivenin (Crotalidae) polyvalent following injection of Crotalus venom. Toxicon 11, 461-464.
Saganuwan, S. A. (2011). A modified method of Reed and Muench for determination of relatively ideal median lethal dose (LD₅₀). Afr. J. Pharm. Pharmaco. 5, 1543-1546.
Sánchez, E. E., Galán, J. A., Pérez, J. C., Rodríguez-Acosta, A., Chase, P. B., Pérez, J. C. (2003). The efficacy of two antivenoms against the venom of North American snakes. Toxicon 41, 357-365.
Sánchez, E. E., Hotle, D., Rodríguez-Acosta, A. (2011). Neutralization of Bitis parviocula (Ethiopian mountain adder) venom by the South African Institute of Medical Research (SAIMR) antivenom. Rev Inst Med Trop Sao Paulo. 53, 213-217.
Sánchez, E. E., Ramirez, M. S., Galán, J. A., López, G., Rodríguez-Acosta, A., Pérez, J. C., Cross reactivity of three antivenoms against North American snake venoms. Toxicon. 41:315-320 (2003).
Smith, T. A. 2d, Figge, H. L. (1991). Treatment of snakebite poisoning. Am J Hosp harm 48, 2190-2196.
Tennant, A., Barlett, R. D. (2000). Snakes of North America: Eastern and Central Regions, Gulf Publishing Company, Houston, pp. 26.
Theakston, R. D. G., Reid, H. A. (1983). Development of simple standard assay procedures for the characterization of snake venoms. Bull. WHO, 61, 949-959.
Tu, Venoms: Chemistry and Molecular Biology, John Wiley & Sons, New York (1977)
Warrell, D. A. (1996). Clinical features of envenoming from snake bites. In: Bon C, Goyffon M, editors. Envenoming and their treatments. Lyon: Fondation Marcel Mérieux. pp. 63-76.
Wingert et al., S. Med. J. 68:1015 (1975)
World Health Organization Publication No. 58 (Geneva 1981)

What is claimed is:

1. A method for treating a venomous bite or sting, comprising administering to a subject suffering from a venomous bite or sting an alkaline sodium silicate composition comprising the empirical formula of Na_{8.2}Si_{4.4}H_{9.7}O_{17.6}.

2. The method of claim 1, wherein the alkaline sodium silicate composition comprises one or more ionizable compounds in equilibrium with the each other.

3. The method of claim 1, wherein the alkaline sodium silicate composition comprises a mixture of:
trimeric sodium silicate (Na₂SiO₃)₃, and
sodium silicate pentahydrate (Na₂SiO₃).5H₂O.

4. The method of claim 3, wherein the sodium silicate pentahydrate (Na₂SiO₃).5H₂O exists in equilibrium in two structural forms of the following general formula:

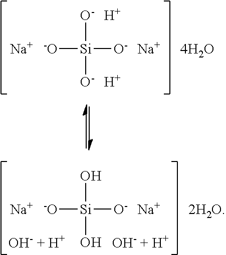

5. The method of claim 1, wherein the venomous bite is from a venomous snake selected from the group consisting of Crotalus atrox, Agkistrodon contortrix contortrix, and Agkistrodon piscivorus leucostoma.

6. The method of claim 1, wherein local hemorrhage and tissue necrosis at or near the site of the venomous bite or sting is reduced.

7. The method of claim 6, wherein the alkaline sodium silicate composition is administered topically at the site of the venomous bite or sting.

8. The method of claim 1, wherein the alkaline sodium silicate composition is administered topically.

9. The method of claim 1, wherein the alkaline sodium silicate composition is administered orally.

10. A method for inhibiting the toxic effects of venom, comprising causing an effective amount of an alkaline sodium silicate composition to come into contact with venom to inhibit the toxic effects thereof, wherein the composition comprises the empirical formula Na_{8.2}Si_{4.4}H_{9.7}O_{17.6}.

11. The method of claim 10, wherein the alkaline sodium silicate composition comprises one or more ionizable compounds in equilibrium with the each other.

12. The method of claim 10, wherein the alkaline sodium silicate composition comprises a mixture of:
trimeric sodium silicate (Na₂SiO₃)₃, and
sodium silicate pentahydrate (Na₂SiO₃).5H₂O.

13. The method of claim 10, wherein the sodium silicate pentahydrate (Na₂SiO₃).5H₂O exists in equilibrium in two structural forms of the following general formula:

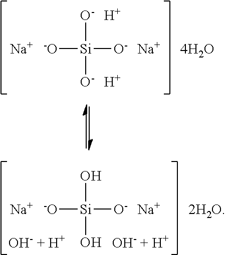

14. The method of claim 10, wherein the venom is from a venomous snake selected from the group consisting of Crotalus atrox, Agkistrodon contortrix contortrix, and Agkistrodon piscivorus leucostoma.
